(19) United States
(12) Patent Application Publication
(10) Pub. No.: US 2004/0088039 A1
(43) Pub. Date: May 6, 2004

(54) Method of securing radiopaque markers to an implant

(76) Inventors: Nathan T. Lee, Golden Valley, MN (US); Victor R. Blackledge, Cologne, MN (US); Paul J. Thompson, Minnetonka, MN (US)

Correspondence Address: MERCHANT & GOULD P.C., P.O. Box 2903, Minneapolis, MN, 55402-0903, US

(21) Appl. No.: 10/286,355

(22) Filed: Nov. 1, 2002

(51) Int. Cl. A61F 2/06

(52) U.S. Cl. 623/1.15; 623/1.34

(57) ABSTRACT

A method for securing a radiopaque marker to an implant is disclosed. The method includes compressing a ball of radiopaque material into an opening defined by the implant.

### TECHNICAL FIELD

This invention pertains generally to medical devices such as stents or other implants. More particularly, the present invention relates to methods for securing radiopaque markers to medical devices such as stents or other implants.

### BACKGROUND

Stents are widely used for supporting a lumen structure in a patient's body. For example, stents may be used to maintain patency of a coronary artery, other blood vessels or other body lumen.

Stents are commonly metal, tubular structures. Stents are passed through a body lumen in a collapsed state. At the point of an obstruction or other deployment site in the body lumen, the stent is expanded to an expanded diameter to support the lumen at the deployment site.

In certain designs, stents are open-celled tubes that are expanded by inflatable balloons at the deployment site. This type of stent is often referred to as a “balloon expandable” stent. Other stents are so-called “self-expanding” stents. Self-expanding stents do not use balloons to cause the expansion of the stent. An example of a self-expanding stent is a tube (e.g., a coil tube or an open-celled tube) made of an elastically deformable material (e.g., a superelastic material such a nitinol). This type of stent is secured to a stent delivery device under tension in a collapsed state. At the deployment site, the stent is released so that internal tension within the stent causes the stent to self-expand to its enlarged diameter. Other self-expanding stents are made of so-called shape-memory metals. Such shape-memory stents experience a phase change at the elevated temperature of the human body. The phase change results in expansion from a collapsed state to an enlarged state.

Stent placement can be visualized through the use of fluoroscopic imaging techniques. These techniques also allow a stent to be viewed during implantation to ensure precise placement of the stent. These techniques also allow the stent to be viewed during post-procedural check-ups to evaluate the condition and effectiveness of the stent.

To improve the fluoroscopic visibility of a stent, it is desirable to increase the radiopacity of the stent. To this end, radiopaque coatings/platings have been applied to stents. A stent having a radiopaque plating is disclosed in U.S. Pat. No. 5,725,572 to Lam et al. Radiopaque markers have also been used to increase the radiopacity of stents. Example stents having radiopaque markers secured thereto are disclosed in U.S. Pat. No. 5,632,771 to Boatman et al., U.S. Pat. No. 6,334,871 to Dor et al., and PCT International Publication No. WO 02/078762.

### SUMMARY

One aspect of the present disclosure relates to a method for securing radiopaque markers to an implant. In one embodiment, a marker is secured to an implant by compressing a ball of radiopaque material into an opening defined by the implant.

Examples of a variety of inventive aspects are set forth in the description that follows. It is to be understood that both the foregoing general description and the following detailed description are exemplary and explanatory only and are not restrictive of the broad inventive aspects disclosed herein.

### BRIEF DESCRIPTION OF THE DRAWINGS

FIG. 1 is a plan view of a one embodiment of a stent shown cut longitudinally and laid flat, the stent includes tips in which radiopaque markers are secured;

FIG. 2 is an enlarged, plan view of one of the tips of the stent of FIG. 1 prior to insertion of a radiopaque marker;

FIG. 3 is a cross-sectional view taken along section line 3-3 of FIG. 2;

FIG. 4 is a cross-sectional view taken along section line 4-4 of FIG. 1;

FIG. 5 is a cross-sectional view taken along section line 5-5 of FIG. 1;

FIG. 6 shows the stent of FIG. 1 mounted on a mandrel with one of the tips of the stent in alignment with a compression anvil;

FIG. 7 shows the stent of FIG. 6 with a radiopaque ball cradled in an opening of the tip;

FIG. 8 shows the stent of FIG. 6 with the anvil lowered such that the radiopaque ball is compressed within the opening of the tip;

FIGS. 9-13 are a sequence of views taken along section line 4-4 of FIG. 1 showing the radiopaque ball of FIGS. 7 and 8 in the process of being compressed within the opening defined by the tip of the stent; and

FIGS. 14-18 are a sequence of views taken along section line 5-5 of FIG. 1 showing the radiopaque ball of FIGS. 7 and 8 in the process of being compressed within the opening defined by the tip of the stent.

### DETAILED DESCRIPTION

With reference now to the various drawing figures in which identical elements are numbered identically throughout, a description is provided of embodiments that are examples of how inventive aspects in accordance with the principles of the present invention may be practiced.

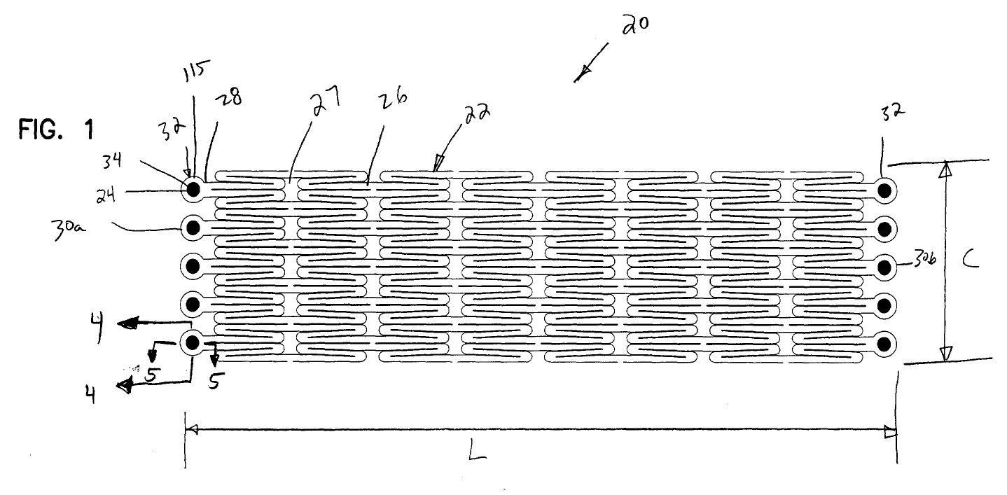

FIG. 1 illustrates a stent 20 including a stent body 22 and a plurality of radiopaque markers 24 secured to the stent body 22. The markers 24 are preferably secured to the stent body 22 by a method in accordance with the present disclosure. As illustrated in the laid flat view of FIG. 1, the stent body 22 defines a length L and a circumference C, and includes a plurality of struts 26 (i.e., reinforcing members). The struts 26 define open cells 27 (i.e., openings) that extend through the stent body 22. The open cells 27 enlarge when the stent 20 expands from an undeployed diameter (shown in FIG. 1) to a deployed diameter (not shown). At least some of the struts 26 have free terminal ends 28 that define proximal and distal ends 30a and 30b of the stent 20. Enlargements 32 are provided at the free terminal ends 28. The enlargements 32 include annular walls 115 (i.e., eyelets) that define openings (i.e., pockets) in the form of through-holes 34. The markers 24 are mounted within the through-holes 34. In alternative embodiments, the openings can be recesses (i.e., depressions) that extend only partially through the stent body 22. A delivery system incorporating the stent 20 is disclosed in U.S. patent application Serial No. not yet assigned, entitled Implant Delivery System with Marker Interlock and having attorney docket No. 11576.68US01, filed on a date concurrent herewith.

The radiopaque markers 24 permit a physician to accurately determine the position of the stent 20 within a patient's lumen under fluoroscopic visualization. The markers 24 are preferably located adjacent the proximal and distal ends 30a, 30b of the stent. Materials for making the radiopaque markers should have a density suitable for visualization through fluoroscopic techniques. In preferred embodiments, the markers have a radiopacity substantially greater than the material used to manufacture the body 22 of the stent 20. Exemplary materials comprise tantalum, iridium, platinum, gold, tungsten and alloys of such metals.

By way of non-limiting, representative example, the stent may be a self-expanding stent having a construction such as that shown in U.S. Pat. No. 6,132,461, which is hereby incorporated by reference in its entirety. In one non-limiting embodiment, the stent can be made of a superelastic metal such as nitinol, or the like. The stent may also be a coil stent or any other self-expanding stent. Another representative stent is shown in U.S. patent application Ser. No. 09/765,725, filed Jan. 18, 2001 and entitled STENT, which is hereby incorporated by reference. It is also contemplated that methods in accordance with the principles of the present disclosure are also applicable to balloon expandable stents. An example material for a balloon expandable stent includes stainless steel. It will be appreciated that the inventive concepts disclosed herein are not limited to the particular stent configuration disclosed herein, but are instead applicable to any number of different stent configurations. For example, the inventive concepts are applicable to stents having a variety of openings, slots or cell shapes and are not limited to the particular cell shapes depicted. Further, while the markers 24 are shown at the ends of the stent 20, it will be appreciated that markers can be mounted at other locations as well.

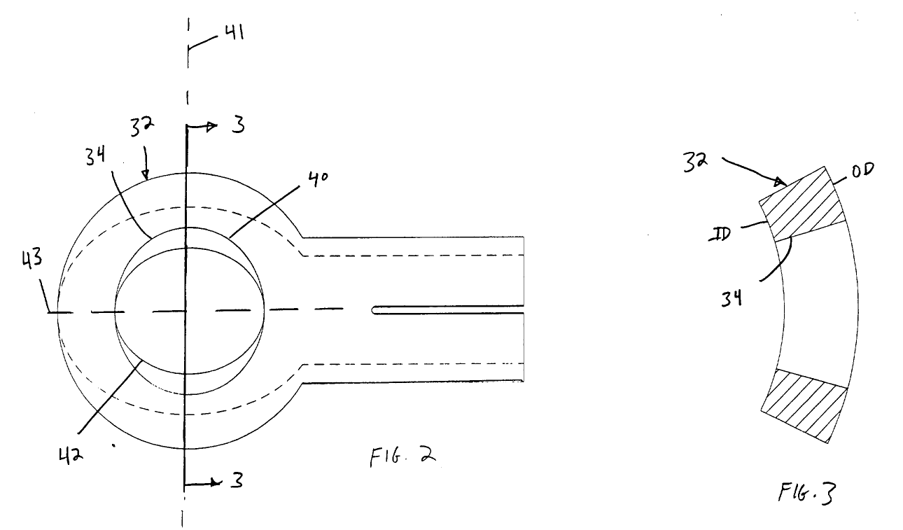

In one embodiment, the stent 12 can be manufactured by cutting (e.g., laser cutting) the open cells 27 and through-holes 34 from a tube of material while leaving the struts 26 intact. It is preferred to cut the through-holes 34 in a generally circular shape. To achieve a generally circular shape taking into consideration the curvatures of the inner diameter ID and the outer diameter OD of the stent body 22, the through-holes are cut with an elliptical shape 40 at the outer diameter and an elliptical shape 42 at the inner diameter ID (see FIG. 2). The shape 40 is elongated along a first axis 41, and the shape 42 is elongated along a second axis 43 that is perpendicular relative to the first axis 41. Interpolating between the inner diameter ID and the outer diameter OD, a generally circular shape is provided generally at a mid-point between the inner and outer diameters. This cutting technique causes the through-hole 34 to taper in such a manner that the cross-sectional area of the through-hole gradually decreases as the through-hole 34 extends from the outer diameter OD toward the inner diameter ID (see FIG. 3). The through-hole 34 thus has a generally truncated cone shape prior to insertion of marker 24.

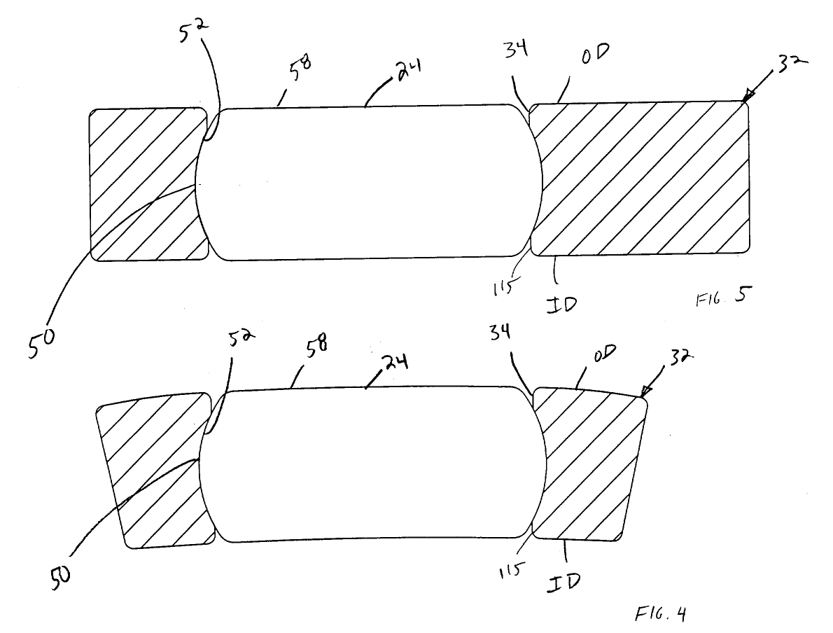

FIGS. 4 and 5 show one of the markers 24 mounted within a corresponding through-hole 34. The marker 24 is compressed within the through-hole 34 and includes an annular projection 50 that extends about the perimeter of the marker 24. The projection 50 projects into a corresponding annular receptacle 52 defined within the wall 115 of the enlargement 32 through which the through-hole 34 extends. The interface between the projection 50 and the receptacle 52 provides an interlock that increases the force required to push the marker from the through-hole 34. In the depicted embodiment, the projection 50 has a convex curvature that extends between the inner diameter ID and the outer diameter OD of the stent body 22, and the receptacle 52 has a complementary concave curvature. The curvatures are preferably provided during the marker insertion process. In one embodiment, the marker 24 has an outer surface 58 that is either flush with or recessed relative to the outer diameter OD of the stent body 22.

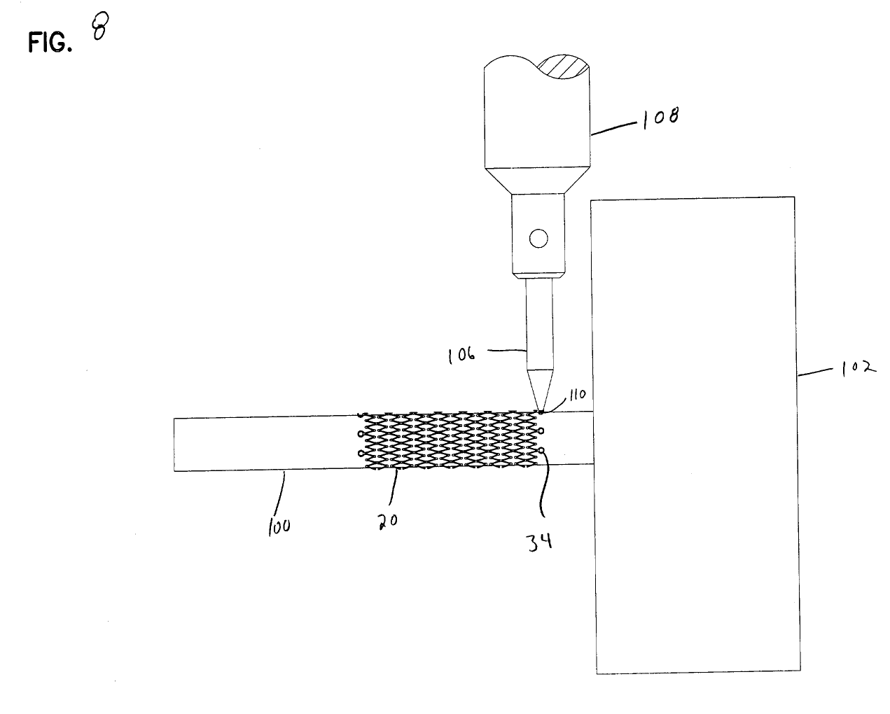

FIGS. 6-8 illustrate an example method for securing one of the markers 24 to the stent body 22. Referring to FIG. 6, the stent body 22 is mounted on a cylindrical mandrel 100. Preferably, the mandrel 100 has an outer diameter that is sized approximately equal to the inner diameter of the stent body 22.

The mandrel 100 is connected to a drive mechanism 102 that rotates or indexes the mandrel about its longitudinal axis. By rotating the mandrel 100 about its longitudinal axis, the through-holes 34 of the stent body 22 can selectively be placed in alignment with a rivet anvil 106. The rivet anvil 106 is coupled to a press 108 that moves the anvil 106 toward and away from the mandrel 100.

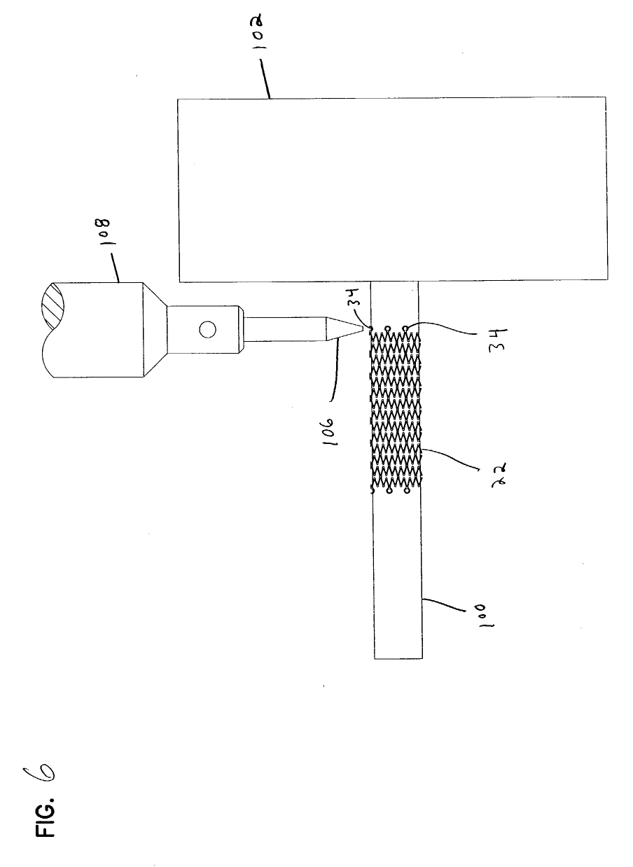

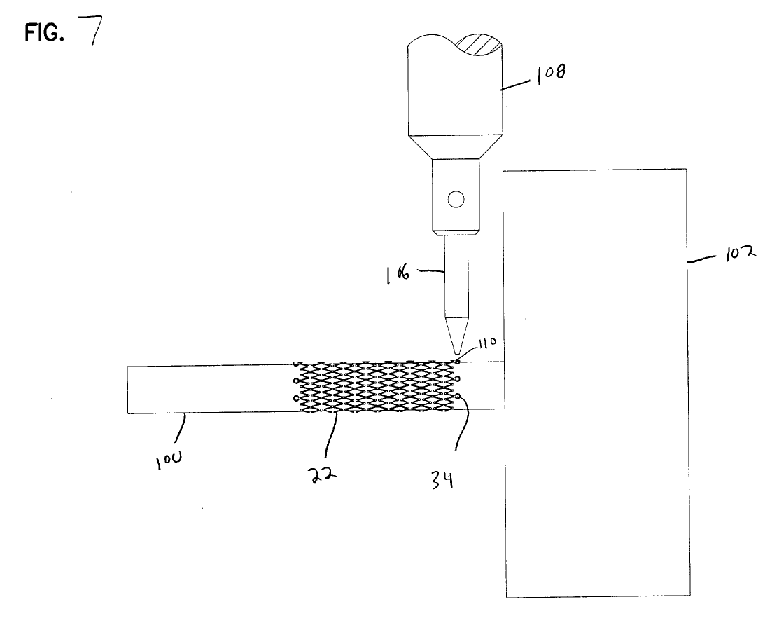

Once the anvil 106 is aligned with a through-hole 34 as shown in FIG. 6, a ball 110 of radiopaque material is placed in the through-hole 34 as shown in FIGS. 7, 9 and 14. The term “ball” means a round or roundish mass or body. Preferably, the ball is spherical in shape. However, the ball could also be oval, elliptical, ovoid or other roundish shapes.

Prior to positioning the ball 110 in the through-hole 34, the ball 100 is preferably cleaned to remove dirt, grease, lapping compounds, abrasive media or any other contaminants. Depending on the type of radiopaque material used, it may be preferred to subject the ball 110 to a bright annealing process to improve the ductility of the ball so as to reduce the likelihood of cracking/fissures during the subsequent compression process. For example, in the case of tantalum, the ball 110 is preferably bright annealed in a 0.0001 Torr or better vacuum oven.

The ball 110 can be positioned in the through-hole 34 by any number of techniques. For example, the ball 110 can be manually placed in the through-hole 34 (e.g., with the aid of a tweezers or other device). Alternatively, the ball 110 can be placed in the through-hole 34 using automated article handling equipment such as dispensing devices (e.g., a funnel arrangement) or vacuum handlers.

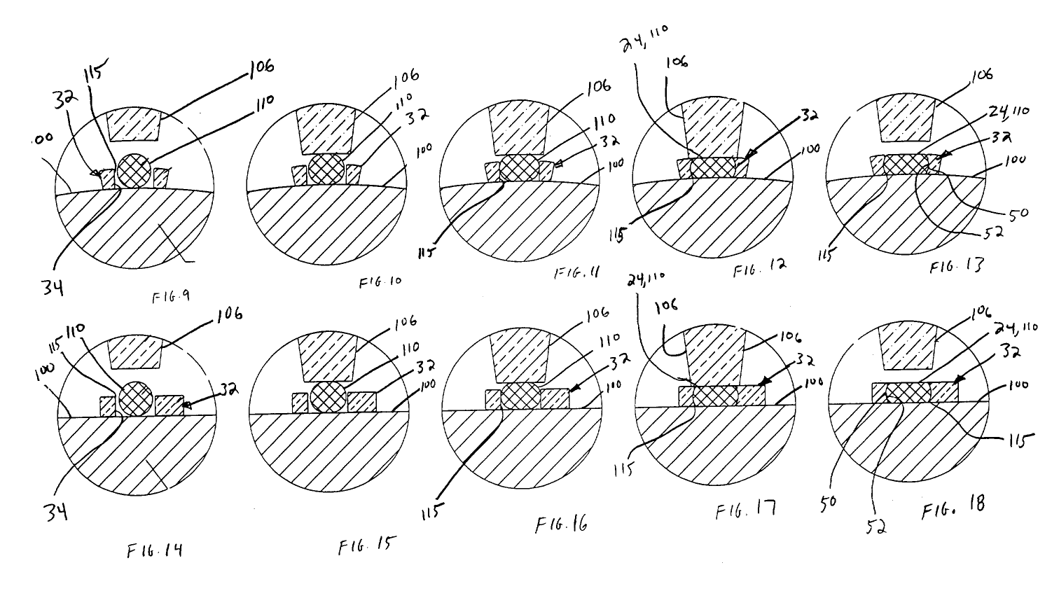

Once the ball 110 is positioned in the through-hole 34, the press 108 is actuated causing the anvil 106 to move toward the mandrel 100. As the anvil 106 moves toward the mandrel 100, the ball 110 is compressed between the tip of the anvil 106 and the outer surface of the mandrel 100. As the ball 110 is compressed, the ball 110 is inelastically deformed (i.e., flattened) as shown in FIGS. 9-13 and 14-18. The flattening of the ball 110 causes the annular wall 115 defining the through-hole 34 to stretch to enable the deformation of the ball 110 within the through-hole 34. As shown in FIGS. 13 and 18, the wall 115 deforms so as to define the annular receptacle 52 that receives the annular projection 50 of the marker 24.

The size of the ball 110 is preferably selected such that the volume of the ball 110 is greater than the volume of the through-hole 34 prior to compression of the ball within the through-hole. However, the ball 110 is preferably sized such that the wall 115 defining the through-hole 34 (i.e., the enlargement 32) does not stretch beyond predetermined limits during compression of the ball 110. For example, in the case of nitinol, it is preferred for the wall 115 to stretch less than 9 percent to reduce the likelihood of failure. Other materials such as stainless steel can stretch greater amounts without failing. Of course, these amounts are merely illustrative and are not intended to limit the scope of the present invention.

After the ball 110 has been compressed within the through-hole 34, the mandrel 110 can be indexed to position the next through-hole 34 in alignment with the anvil 106. Thereafter, the process can be repeated until all of the through-holes 34 are filled with markers 24.

The use of balls as rivets provides numerous advantages. For example, it has been determined by the inventors that the riveting or compression of radiopaque balls within a wall of an implant yields markers having excellent retention characteristics. Also, in the case of spherical balls, the balls can be manufactured to tight tolerances thereby providing accurate volumetric control over the radiopaque material. This results in a repeatable, consistent riveting process. Spherical balls can also be readily finished using precise finishing techniques. Moreover, spherical balls facilitate automation because the balls need not be inserted into the through-holes in any particular orientation.

While the various embodiments of the present invention have related to stents, the scope of the present invention is not so limited. By way of non-limiting example, other types of implants include anastomosis devices, blood filters, grafts, vena cava filters, percutaneous valves, or other devices.

It has been shown how the objects of the invention have been attained in a preferred manner. Modifications and equivalents of the disclosed concepts are intended to be included within the scope of the claims.

What is claimed is:

1. A method for mounting a radiopaque marker to an implant, the method comprising:
compressing a ball of radiopaque material into an opening defined by the implant.

2. The method of claim 1, wherein the ball is inelastically deformed when compressed within the opening.

3. The method of claim 2, wherein in the ball is sphere-shaped prior to being compressed within the opening.

4. The method of claim 1, wherein the opening is non-ball shaped.

5. The method of claim 1, wherein the implant has a tubular configuration including a reinforcing structure defining inner and outer diameters, wherein the opening extends at least partially through the reinforcing structure of the implant.

6. The method of claim 5, wherein the opening extends completely through the reinforcing structure from the outer diameter to the inner diameter.

7. The method of claim 6, wherein the opening includes a cross-sectional area that continuously decreases in size as the opening extends from the outer diameter to the inner diameter.

8. The method of claim 7, wherein the ball is compressed into the opening in a direction extending from the outer diameter toward the inner diameter.

9. The method of claim 8, wherein as the ball is compressed into the opening, the implant is supported by a support member located inside the inner diameter of the implant.

10. The method of claim 9, wherein the ball is pressed against the support member during compression of the ball.

11. The method of claim 1, wherein the ball is annealed prior to compressing the ball into the opening.

12. The method of claim 11, wherein the radiopaque material includes tantalum.

13. The method of claim 1, wherein the radiopaque material is selected from a group of materials including iridium, tantalum, platinum, gold, tungsten, or alloys thereof.

14. The method of claim 1, wherein the implant is a stent.

15. The method of claim 14, wherein the stent defines a plurality of openings into which balls of radiopaque material are compressed.

16. The method of claim 15, wherein the stent includes ends having tips, wherein at least some of the tips include enlargements, and wherein the openings are defined through the enlargements.

17. The method of claim 1, wherein the ball is annealed prior to being compressed in the opening.

18. A method for mounting a radiopaque marker to an implant, the method comprising:
compressing a sphere of radiopaque material into a non-spherical opening defined by the implant thereby causing the sphere to inelastically deform within the opening.

19. The method of claim 18, wherein the implant has a tubular configuration including a reinforcing structure defining inner and outer diameters, wherein the opening extends completely through the reinforcing structure from the outer diameter to the inner diameter, and wherein the opening includes a cross-sectional area that continuously decreases in size as the opening extends through the reinforcing structure.

20. The method of claim 19, wherein the cross-sectional area of the opening continuously decreases in size as the opening extends through the reinforcing structure from the outer diameter to the inner diameter, and wherein the sphere is compressed into the opening in a direction extending from the outer diameter toward the inner diameter.

21. The method of claim 20, wherein as the sphere is compressed into the opening, the implant is supported by a support member located inside the inner diameter of the implant, and the sphere is pressed against the support member during compression of the sphere.

22. The method of claim 18, wherein the sphere is annealed prior to being compressed in the opening.
